(19) United States
(12) Patent Application Publication
(10) Pub. No.: US 2012/0065713 A1
(43) Pub. Date: Mar. 15, 2012

(54) THERMO-ELECTRIC DEVICE

(75) Inventors: Matthew Campbell Greaves, Queensland (AU); Michele Sterling, Queensland (AU)

(73) Assignee: The University of Queensland, Queensland (AU)

(21) Appl. No.: 13/319,573

(22) PCT Filed: May 11, 2010
(86) PCT No.: PCT/AU10/00547

§ 371 Date: Nov. 9, 2011

(30) Foreign Application Priority Data
May 11, 2009 (AU) 2009902080

(51) Int. Cl. A61F 7/00

(52) U.S. Cl. 607/96

(57) ABSTRACT

A portable handheld thermo-electric device (10) is used to test for decreased cold pain thresholds or increased sensitivity to cold stimulation (cold hyperalgesia) in a patient. The thermo-electric device (10) comprises a probe (18), a Peltier module (12) and a control unit (16) operable to energize the Peltier module (12) so that the temperature of the probe 18 is variable in a range between a predetermined upper temperature limit and a predetermined lower temperature limit during a test cycle of the thermo-electric device (10). A thermal mass (14) is adapted for thermal contact with an interface side (34) of the Peltier module (12). The Peltier module (12) has thermal properties which enables it to be cooled to a temperature below the predetermined upper temperature limit and thereafter maintain the interface side (34) of the Peltier module (12) at a temperature below the predetermined upper temperature limit for the duration of the test cycle.

### FIELD OF THE INVENTION

The invention relates to a thermo-electric device. In particular, although not exclusively, the invention relates to a portable handheld thermo-electric device for affecting a temperature drop on the skin as an indicator of decreased cold pain thresholds or increased sensitivity to cold stimulation (cold hyperalgesia) in a patient.

### BACKGROUND TO THE INVENTION

Cold hyperalgesia (or decreased cold pain threshold) is a feature of some musculoskeletal pain conditions and is thought to reflect changes in central nervous system pain processing mechanisms. In the case of whiplash injury (that is neck pain following a motor vehicle crash) the presence of cold hyperalgesia is important as this feature has been shown to be predictive of poor functional recovery and be associated with non-responsiveness to standard physical interventions, e.g. exercise.

The early identification of whiplash injured people at risk of poor recovery is important for several reasons, including: the institution of early and appropriate interventions; the possible need to involve an interdisciplinary team of health care providers including medical practitioners, physiotherapists, and psychologists; to allow insurance bodies to provide appropriate and adequate funding for the specific treatment needs of claimants and to decrease insurance costs via improving recovery rates or facilitating earlier claim settlements.

As a result of recent findings, the Australian Guidelines for Whiplash Management (MAA, 2007) now recommend that clinicians include measures of cold hyperalgesia in the assessment of patients with whiplash. At the present time this is not possible in the primary care environment in which the vast majority of people with whiplash injury are managed.

Peltier modules are solid state devices which work as “heat pumps” to move heat from one side of the module to the other side when a DC current is applied over the module. The flow of heat is reversed when the current is reversed so that one side of the Peltier module can be used both as a heater or a cooler depending on the polarity of the applied current. Peltier modules are not very energy efficient as they draw high amounts of power to provide a large temperature differential between their sides. Peltier modules have not been considered suitable on their own for cold hyperalgesia testing as the temperature range used in cold hyperalgesia testing would require that the temperature differential of the Peltier module be so large as to require impractical amounts of power to effectively energise the Peltier module.

U.S. Pat. No. 5,191,896, assigned to Medoc Ltd, teaches an apparatus for measuring threshold sensitivity to a stimulus, including a separate cooling unit, probe unit and computer. The threshold sensitivities measured are warm sensation, cold sensation, hot pain and cold pain. The probe unit includes Peltier devices for heating and cooling. The probe unit is connected to the cooling unit by conduits through which cooling fluid flows. In use, heat exchangers connected to the Peltier devices in the probe unit are maintained at a generally constant temperature such as 32° C. through operation of the cooling unit which circulates the cooling fluid through the heat exchangers.

Because the apparatus of U.S. Pat. No. 5,191,896 comprises a number of separate spaced apart components the apparatus is generally bulky and not suitable for easy transportation. Connecting the cooling unit to the probe unit by the flow of cooling fluid between the units is complex and lends itself to mechanical breakdown. Additionally the units are expensive and this is not feasible for use in the clinical environment.

### DISCLOSURE OF THE INVENTION

In one form, although it need not be the only or indeed the broadest form, the invention resides in a thermo-electric device comprising:

a probe;

a Peltier module having a probe side in thermal contact with the probe and an opposite interface side;

a control unit electrically connected to the Peltier module and connectable to a power source to energize the Peltier module so that the temperature of the probe side of the Peltier module, and thus the temperature of the probe, is variable in a range between a predetermined upper temperature limit and a predetermined lower temperature limit during a test cycle of the thermo-electric device; and

a thermal mass adapted for thermal contact with the interface side of the Peltier module and having thermal properties which enables it to be cooled to a temperature below the predetermined upper temperature limit and thereafter maintain a temperature below the predetermined upper temperature limit for the duration of the test cycle.

The thermal properties of the thermal mass preferably enables it to maintain the interface side of the Peltier module at a temperature between the predetermined upper temperature limit and the predetermined lower temperature limit for the duration of the test cycle without being cooled during the test cycle. The thermal mass preferably has a volumetric heat capacity of more than 2.00 J/cm³ K¹.

The control unit is configured to energise the Peltier module to heat the probe for part of the test cycle so that the probe is at a temperature warmer than the thermal mass, and to energise the Peltier module for a different part of the test cycle to cool the probe to a temperature cooler than the thermal mass. The Peltier module is preferably first energised to heat the probe relative to the thermal mass and then to cool the probe relative to the thermal mass.

The control unit preferably includes a temperature sensor at the thermal mass to measure the temperature of the thermal mass before the start of the test cycle. A visual or audible signal device preferably signals to a user of the thermo-electric device that the thermal mass has been cooled to below a threshold temperature, of for example 10 degrees Celsius. The control unit is preferably configured to allow the thermo-electric device to start the test cycle only if the temperature of the thermal mass is measured to be below the threshold temperature.

The thermo-electric device may include a housing in which the thermal mass is located and at least part of the thermal mass is exposed to allow heat transfer of the thermal mass with a cold block during cooling of the thermal mass. The thermal mass may be removably located in the housing so that it may be removed for cooling. The thermo-electric device is preferably a unitary handheld device.

The thermo-electric device may include a secondary Peltier module having a cold face which is in thermal contact with the thermal mass and which brings the thermal mass to a temperature between the predetermined upper temperature limit and the predetermined lower temperature limit before the start of the test cycle.

The power source may be mains power, or the thermo-electric device may include a rechargeable battery as the power source.

In another form, the invention resides in a thermo-electric system comprising the thermo-electric device as defined and described hereinabove and a base stand having a selectively cooled cold block which the thermal mass contacts during cooling of the thermal mass.

The invention extends to a thermo-electric device kit comprising the thermo-electric device as defined and described hereinabove and a cold block in the form of a block of material on which the thermal mass is placed during cooling by heat transfer between the thermal mass and the block of material.

In still another form, the invention resides in a method of thermal control of a thereto-electric device, the method including:

cooling a thermal mass of the thermo-electric device to a temperature below a predetermined upper temperature limit;

ceasing cooling of the thermal mass before the start of a test cycle of the thermo-electric device and for the duration of the test cycle; and

energising a Peltier module of the thermo-electric device so that a probe of the thermo-electric device, which is in thermal contact with the Peltier module, is variable between the predetermined upper temperature limit and a predetermined lower temperature limit during the test cycle. The thermal mass is preferably cooled to a temperature between the predetermined upper temperature limit and the predetermined lower temperature limit before the test cycle. The method preferably includes measuring the temperature of the thermal mass before the start of the test cycle to determine whether the thermal mass is below a threshold temperature required for the test cycle.

The Peltier module is preferably energised to heat the probe for part of the test cycle so that the probe is at a temperature warmer than the thermal mass, and the Peltier module is energised for a different part of the test cycle to cool the probe to a temperature cooler than the thermal mass. The Peltier module is preferably first energised to heat the probe relative to the thermal mass and then energised to cool the probe relative to the thermal mass.

The thermal mass is preferably cooled by putting the thermal mass into contact with a cold block so that the thermal mass is cooled by heat transfer between the thermal mass and the cold block. The thermal mass may be cooled by a second Peltier module abutting the thermal mass.

The thermal mass may be located in a housing of the thermo-electric device with at least part of the thermal mass exposed to allow contact with the cold block and removing the thermal mass from the cold block comprises removing the thermo-electric device from the cold block. Alternatively, the thermal mass is removably located in the housing and the thermal mass is removed from the housing for cooling and inserted back into the housing after cooling.

The predetermined upper temperature may be a temperature between 50 degrees Celsius and 25 degrees Celsius and preferably a temperature between 35 degrees Celsius and 25 degrees Celsius. The predetermined lower temperature limit may be a temperature between 0 degrees Celsius and 8 degrees Celsius and preferably be between 0 degrees Celsius and 5 degrees Celsius. The threshold temperature to which the thermal mass is cooled before the start of the test cycle is preferably below 15 degrees Celsius.

Further features of the present invention will become apparent from the following detailed description.

### BRIEF DESCRIPTION OF THE DRAWINGS

To assist in understanding the invention and to enable a person skilled in the art to put the invention into practical effect preferred embodiments of the invention will be described by way of example only with reference to the accompanying drawings, wherein:

FIG. 1 shows a perspective top view of one embodiment of a thermo-electric device in accordance with the invention;

FIG. 2 shows a perspective bottom view of the thermo-electric device of FIG. 1;

FIG. 3 shows a perspective view of a thermo-electric device kit comprising the thermo-electric device of FIG. 1 and a cold block on which the thermo-electric device is placed to cool a thermal mass of the thermo-electric device;

FIG. 4 shows a sectional side view of the thermo-electric device of FIG. 1;

FIG. 5 shows a thermo-electric system in accordance with the invention, including a base stand and a the thermo-electric device;

FIG. 6 shows a cross sectional view of the thermo-electric device of FIG. 5;

FIG. 7 shows yet another embodiment of a thermo-electric device in accordance with the invention; and

FIG. 8 is a flow diagram of a method of thermal control of the thermo-electric device of FIG. 1.

### DETAILED DESCRIPTION OF THE INVENTION

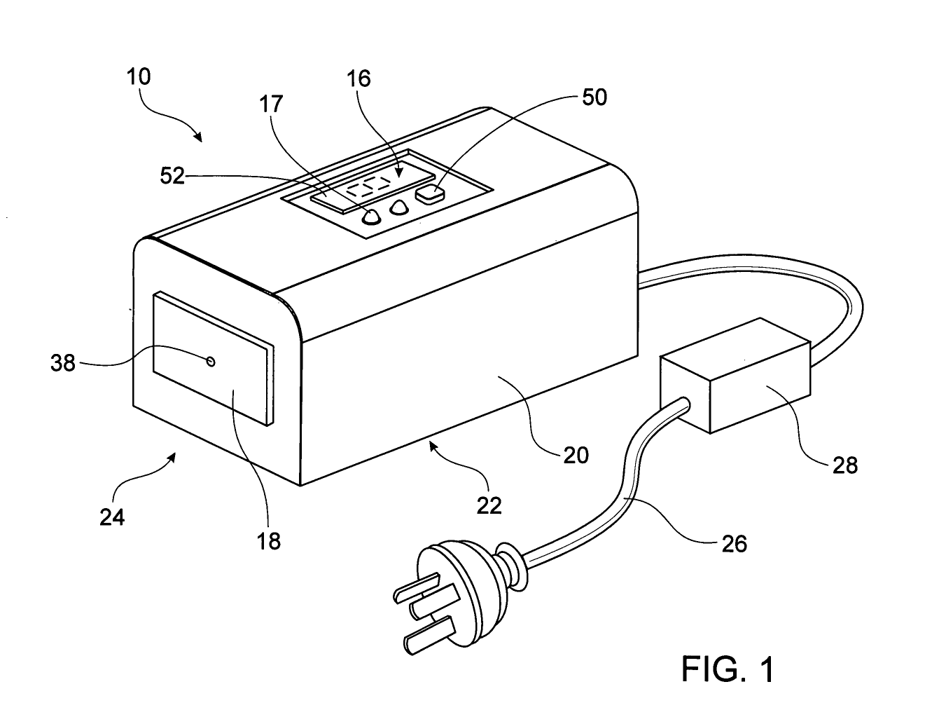

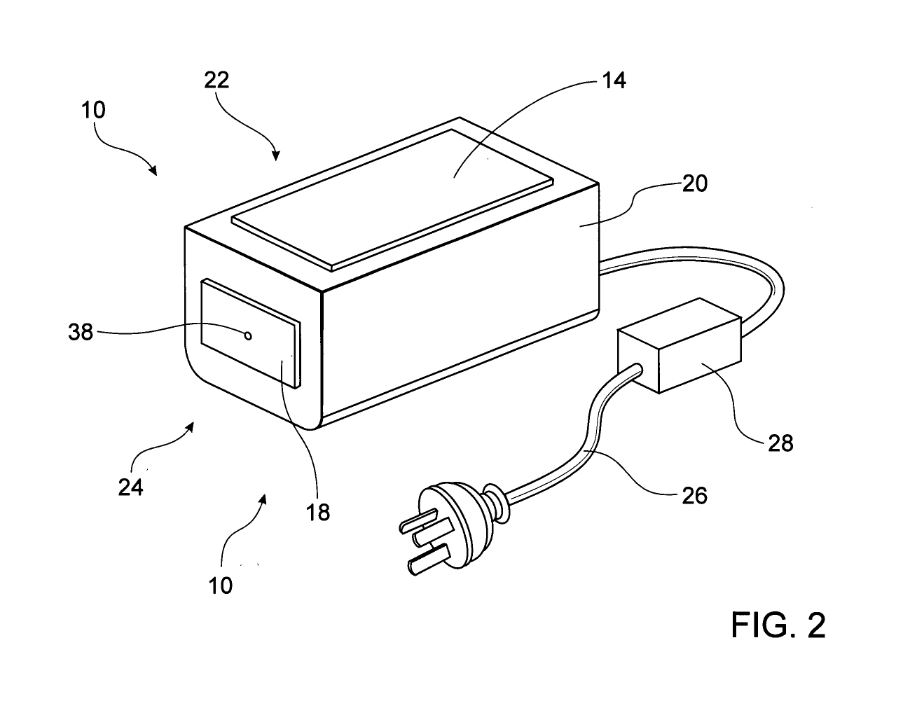

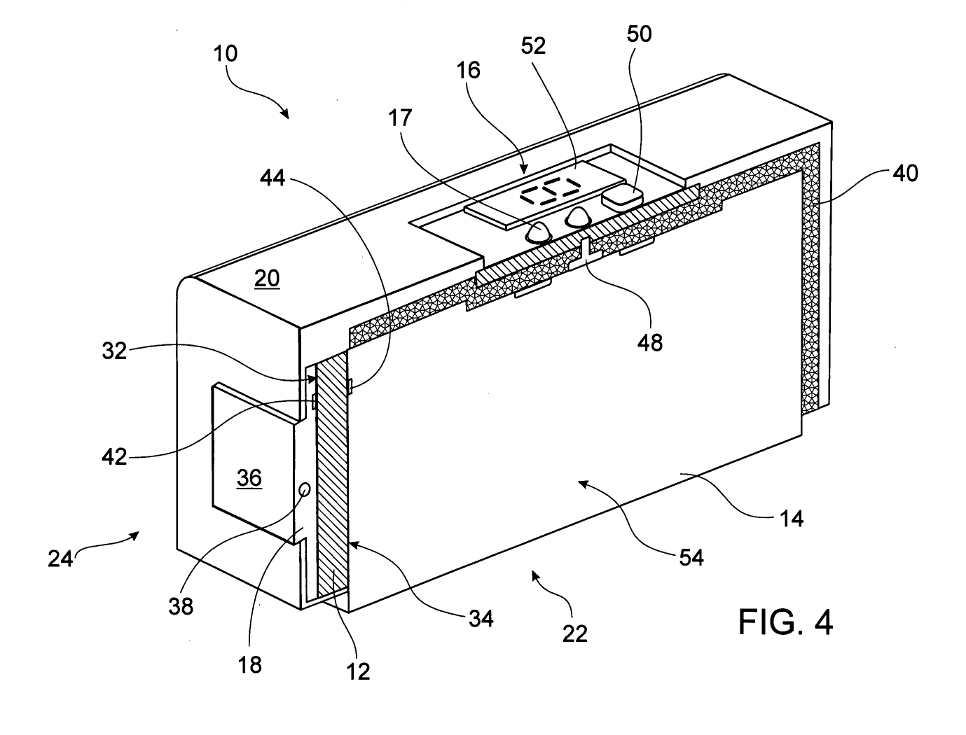

With reference to FIGS. 1 to 4 of the drawings, one embodiment of a thermo-electric device in accordance with the invention is designated generally by reference numeral 10. The thermo-electric device 10 is a unitary handheld device used for cold hyperalgesia testing. The thermo-electric device 10 includes a thermal mass 14, a control unit 16, a probe 18 and a housing 20. A Peltier module 12 (shown in FIG. 4) is located between the thermal mass 14 and the probe 18.

The thermal mass 14 is exposed at an underside 22 of the device 10 and the probe 18 is exposed at a front end 24 of the device 10. The thermal mass 14 and probe 18 stand proud from the housing 20.

Electric power is supplied to the control unit 16 from mains electricity via a power cord 26 and a transformer 28.

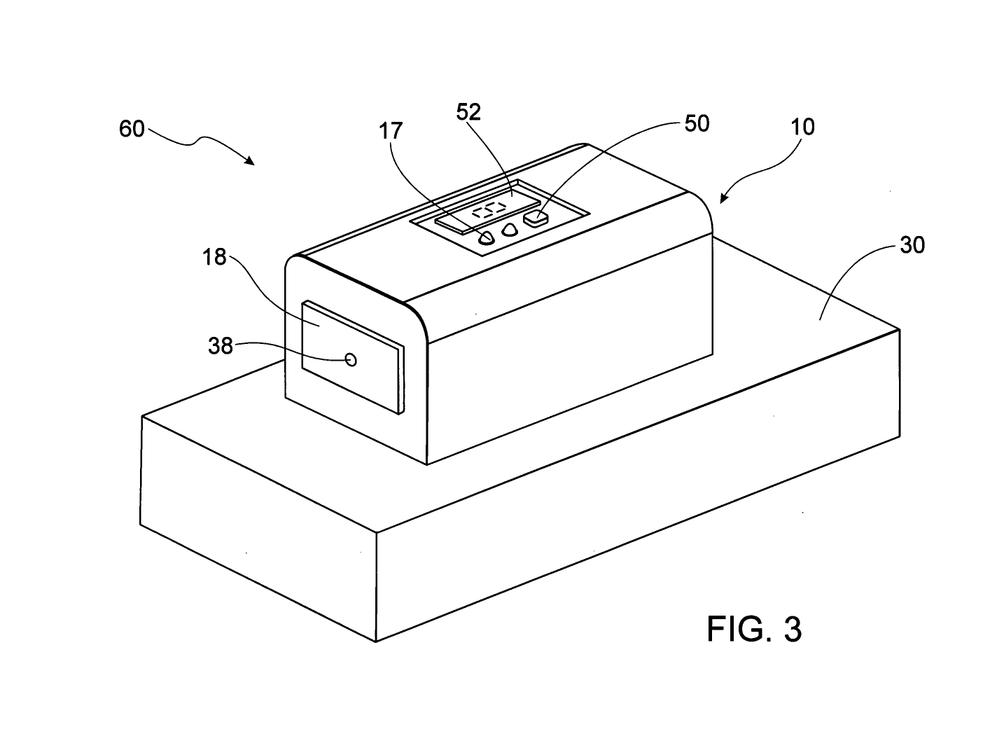

Referring to FIG. 3, the thermal mass 14 is cooled by placing the device 10 on a cold block 30. The cold block 30 is a block of aluminium material. The cold block 30 is pre-cooled by being placed in a freezer or the like and then taken out of the freezer for cooling the thermal mass 14. The thermal mass 14 is cooled by heat transfer between the cold block 30 and the thermal mass 14 when the device 10 is placed on the cold block 30.

The control unit 16 has a visual indicator in the form of an LED lamp 17 which glows constant green when the control unit 16 detects the thermal mass 14 to be cooled down to a threshold temperature. In another embodiment the LED lamp 17 is substituted by an audible alarm which sounds when the control unit 16 detects the thermal mass 14 to be cooled down to a threshold temperature. The threshold temperature is typically between 8° and 12° C. It will be appreciated that the threshold temperature set in the control unit 16 may be different for different sizes and types of thermal masses.

The components and working of the device 10 is described with reference to FIG. 4. The Peltier module 12 is located between the thermal mass 14 and the probe 18. The Peltier module 12 has a probe side 32 and an interface side 34. The probe side 32 abuts against the probe 18 and the interface side 34 abuts against the thermal mass 14. Thermally conductive paste or bonding agent is preferably put between the Peltier module 12 and the probe 18 and between the Peltier module 12 and the thermal mass 14. The thermally conductive paste promotes thermal contact between the components.

The Peltier module 12 is energised by the control unit 16 to control the temperature of the probe 18 during a test cycle between a predetermined upper temperature limit and a predetermined lower temperature limit. When electrical current is passed through the Peltier module 12 the temperature of one of the sides 32 or 34 is raised and the temperature of the other side 32 or 34 is lowered. Which side, 32 or 34, has the temperature rise or lowering is dependant on the polarity of the voltage applied across the Peltier module 12. The control unit 16 is connected to the Peltier module 12 by electric wires (not shown) which have contacts at the opposite sides 32, 34 of the Peltier module 12. The control unit 16 is operable to change the polarity of the voltage applied across the Peltier module 12 during the test cycle, and thus control if the Peltier module is energised as a heater or cooler of the probe 18 relative to the thermal mass 14.

The thermal mass 14 abuts the interface side 34 of the Peltier module 12. The function of the thermal mass 14 is to keep the interface side 34 at or close to the threshold temperature during the test cycle by heat transfer with the Peltier module 12. The less the temperature differential between the sides 32, 34 of the Peltier module 12 during the test cycle, the less energy the Peltier module 12 uses. The threshold temperature is thus chosen to be a temperature between the upper temperature limit and lower temperature limit so that the Peltier module 12 alternatively heats and then cools the probe 18 during the test cycle. The Peltier module 12 is generally more efficient at heating than cooling, so the threshold temperature is normally below the mean between the upper temperature limit and lower temperature limit. Because the Peltier module 12 is generally more efficient at heating than cooling, the thermal mass 14 may be cooled to a temperature below the lower temperature limit and still operate effectively. In certain instances the threshold temperature may thus be lower than the lower temperature limit.

During an example single test cycle of cold hyperalgesia testing, the probe 18 is linearly cooled from an upper temperature limit of 30° C. to a lower temperature limit of 5° C. at a rate of approximately 1° C. per second. In order for the Peltier module 12 to not use excessive amounts of power, the thermal mass 14 has to be cooled to a temperature between the upper temperature limit and the lower temperature limit before the start of the test cycle. The thermal mass 14 is typically cooled to between 8° C. and 12° C. before the start of the test cycle. The threshold temperature set in the control unit 16 may thus be any one of 8° C. 9° C. 10° C. 11° C. and 12° C. Cooling of the thermal mass 14 before testing may be assisted by running the Peltier module 12 as a cooler for the thermal mass 14 before the start of the test cycle. Although the example is described by reference to an upper temperature limit of 30° C. and a lower temperature limit of 5° C., the upper temperature limit may be as high as 50° C. and as low as 25° C. The lower temperature limit may be as high as 8° C. and as low as 0° C. The threshold temperature set in the control unit 16 will be suitably adapted for such other upper temperature limits and lower temperature limits as required.

The probe 18 has a face 36 which is rectangular in plan view and dimensioned for contact with the back of the necks of whiplash patients during cold hyperalgesia testing. A temperature sensor 38 of the control unit 16 is embedded in the probe 18. The temperature sensor 38 measures the temperature of the probe 18 and thus of the face 36. The probe 18 is of a material having high thermal conductivity, such as aluminium. The probe 18 optimally has a low thermal inertia, by being of only enough thickness to contain the temperature sensor 38.

The thermal mass 14 is of a material having a relatively high specific heat capacity and a relatively high volumetric heat capacity, for example aluminium. Aluminium has a specific heat capacity of 0.897 J/gK (at 25° C.) and a volumetric heat capacity of 2.422 J/cm³ K¹ (at 25° C.). The thermal mass is preferably also of a material which is a good conductor of heat, and thus has a relatively high thermal conductivity k. Aluminium has a thermal conductivity k value of 237 W/(m·K) at 25° C. Aluminium alloys have a k value of between 120 and 180. The Applicant envisages that the thermal conductivity k value for the thermal mass 14 would have to be above at least 10 (at 25° C.) to allow for effective functioning of the device 10. The thermal mass 14 will typically be a 40 mm×40 mm×80 mm rectangular block. The thermal mass 14 is additionally insulated with insulating material 40 which partially surrounds the thermal mass 14. The insulating material 40 insulates the thermal mass 14 against absorption of ambient heat. Before the start of the test cycle, the thermal mass 14 is cooled to the threshold temperature or below by placing it on the cold block 30.

The thermal properties of the thermal mass 14 is central to the invention but the size, shape and actual material type of the thermal mass may vary while still having the thermal properties which enables the effective functioning of the thermo-electric device 10. In particular, although not exclusively, the thermal mass 14 material may comprise magnesium, copper, zinc alloy, titanium, aluminium alloy, or brass. In another embodiment of the thermal mass 14, the thermal mass is in the form of a liquid filled container and more specifically the container may be filled with oil or primarily with water. The container may have conductive internal fins to promote thermal conductivity from the oil or water to the probe 18. The thermal mass 14 may thus be a metal matrix composite or a hybrid thermal mass. The thermal mass 14 may be a polymer as is known for cold packs, which could be adapted to include a metal film, such as aluminium foil to aid in heat transfer to the Peltier Module 12. The thermal mass 14 preferably has a relatively high volumetric heat capacity and a relatively high thermal conductivity. The cold block 30 may be of the same material as the thermal mass 14.

The control unit 16 includes a logic controller for controlling the device 10. The control unit 16 has contacts 42, 44 at the sides 32, 34 of the probe 18, through which a voltage is placed across the Peltier module 12 to energize the Peltier module 12. The control unit 16 includes a proportional-integral-derivative (PID) controller which regulates the voltage and current placed across the Peltier module 12 as will be described in more detail herein below. Temperature measured by the temperature sensor 38 is a process variable input to the PID controller. The PID controller regulates the voltage and current placed across the Peltier module 12 so that during a single test cycle the temperature of the probe portion 20 linearly cools from a staring temperature of 30° C. to a lower temperature limit of 5° C. at a rate of approximately 1° C. per second.

The control unit 16 includes a thermal mass temperature sensor 48. The temperature sensor 48 is an input to the control unit 16 in order for the control unit 16 to measure if the thermal mass 14 is cooled to the threshold temperature or below. If the thermal mass 14 is at the threshold temperature or below, the LED lamp 17 is energised to be a constant green, thereby to indicate to a user of the device 10 that the device is ready to start a test cycle. The control unit 16 also includes a start/stop/reset button 50. The button 50 is operated by the user to start the test cycle, stop the test cycle or reset the device 10.

The control unit 16 further includes a digital display 52 which displays the temperature measured by the temperature sensor 38, which is indicative of the temperature of the probe 18 and thus the temperature at the skin of a patient the probe 18 is applied to.

In another embodiment of the invention, the thermal mass 14 of the device 10 is removably located in the housing 20. The housing has a cavity 54 for removably receiving the thermal mass. The thermal mass may slot into and out of the cavity 54. The cavity 54 may be adapted for receiving a gel cold pack. The thermal mass 14 is removed to be separately cooled and then placed back into the housing 20 after cooling. The thermal mass 14 will typically be cooled by being placed in a fridge, freezer or the like.

The thermo-electric device 10 is used for measurement of cold pain thresholds of whiplash patients. The device 10 may, however be used for other types of temperature stimulus testing as is known in the field of neuropathy. It has been found that patients with whiplash injuries have decreased cold pain threshold or increased sensitivity to cold stimulation of the neck. In order to assess cold pain threshold of patients, the probe 18 is applied against the skin of a patient at the start of a test cycle. As a first step, before the start of a test cycle the thermal mass 14 is cooled down to the threshold temperature of 10° C. The thermal mass 14 is cooled down by taking the cold block 30 out of the freezer and placing the device 10 on the cold block 30. The thermal mass 14 cools by heat transfer between the cold block 30 and the thermal mass 14. The LED 17 lights up when the thermal mass 14 is sufficiently cooled to be at or below 10° C. The device 10 heats the probe 18 to the upper temperature limit of 30° C. during cooling of the block 30 and maintains it at 30° C. before the start of the test cycle. To start the test cycle, the button 50 is pushed and the control unit 16 drops the temperature at a specified rate, for example 1° C. per second. Once the patient indicates that he/she is experiencing pain because of the applied face 36, the probe 18 is removed from contact with the skin and the temperature of the probe 18 when the patient experienced pain is recorded. The button 50 can be pushed again to reset the test cycle. If the thermal mass 14 is still below 10° C. the control unit 16 will allow for another test cycle.

The device 10 may be provided with a push button connected to the control unit 16, which the patient presses as soon as he/she experiences pain and the control unit 16 will be operable to record and display the temperature at which the patient pressed the push button. It has been found that if pain is experienced at a face temperature of higher than approximately 15° C. it is indicative of poor recovery whiplash trauma to the neck of the patient.

At the upper temperature limit of 30° C. the Peltier module 12 works as heater in that the probe 18 is heated relative to the thermal mass 14. At the lower temperature limit of 5° C. the Peltier module 12 works as a cooler in that the probe 18 is cooled relative to the thermal mass 14. The thermal mass 14 heats up during the test cycle, but remains at between 8° C. and 16° C. by the selection of an appropriately sized thermal mass 14. In between the upper and the lower temperature limits the Peltier module 12 transitions from being a heater to a cooler. At the transition of the Peltier module 12 from a heater to a cooler the polarity of the voltage applied over the Peltier module 12 is reversed.

The temperature differential between the sides 32, 34 of the Peltier module 12 is kept relatively low by the thermal mass 14 being able to hold the interface side 34 at or about 10° C. This allows the Peltier module 12 to operate at a higher coefficient of performance than if the thermal mass was removed and also to operate with less thermal stresses on the Peltier module 12. The Peltier module 12 does not consume excessive amounts of power and the thermo-electric device 10 is able to be built as a portable and compact unit because of the thermal mass 10. Peltier modules are also less prone to failing due to temperature stresses if the temperature differentials between their opposite sides are kept as low as possible, thus to limit heat stresses in the Peltier modules. Peltier modules that have specifications for lower temperature differentials are also cheaper. Peltier devices have high rates of thermal change which makes them well adapted to heat and cool the probe 18 for cold hyperalgesia testing.

FIG. 3 shows a thermo-electric device kit 60 in accordance with the invention comprising the device 10 and the cold block 30.

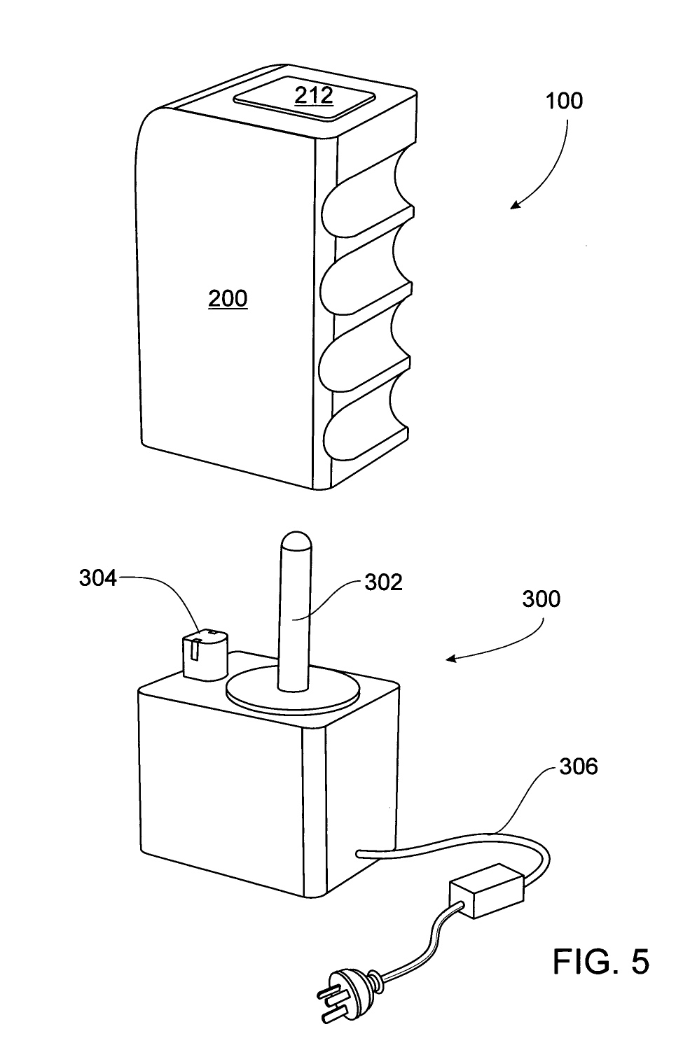

FIG. 5 shows a thermo-electric system 100 in accordance with the invention comprising a thermo-electric device 200 and a base stand 300.

The base stand 300 has a cold block in the form of a spigot 302. The spigot 302 stands operatively upright. The base stand 300 also includes an electrical connector 304 having positive and negative electric contacts. The base stand 300 is powered by mains electricity via a power cord 306.

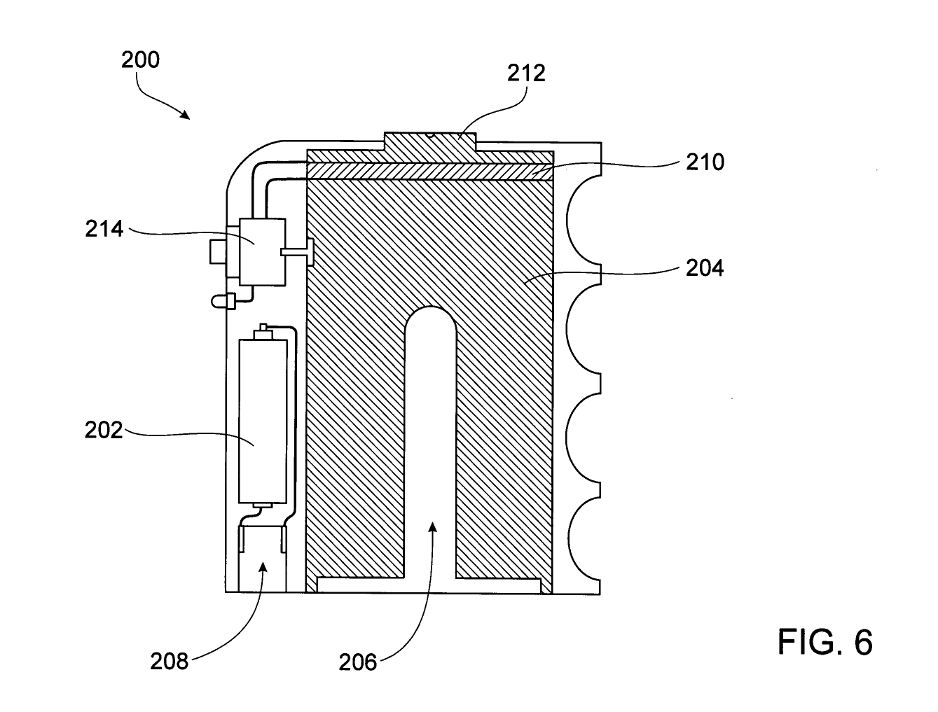

The base stand 300 includes a cooling system (not shown) for cooling the spigot 302. The cooling system may continuously cool the spigot 302 or selectively rapidly cool the spigot 302. The device 200 is seated on the base stand 300 to cool a thermal mass of the device 200 and charge batteries of the device 200 as is described with reference to FIG. 6.

FIG. 6 shows a sectional view of the device 200. The device 200 is similar to the device 10, except that it includes rechargeable battery pack 202 as a power source and the thermal mass 204 has a passage in the form of a hole 206 for receiving the spigot 302 of the base stand 300. The battery pack 202 is charged when the device 200 is seated on the base stand 300. The electrical connector 304 is received in a socket 208 of the device 200 to charge the battery pack 202.

The thermal mass 204 of the device 200 is cooled by the spigot 302 of the base stand 300 in the same manner as the thermal mass 14 of the device 10 is cooled by being put into contact with the cold block 30. The device 200 includes a Peltier module 210, a probe 212 and a control unit 214, which are similar to the components described for the device 10. The device 200 works the same as the device 10, except that it is fully portable in that it includes the rechargeable battery pack 202 for energising the Peltier module 210 during a test cycle.

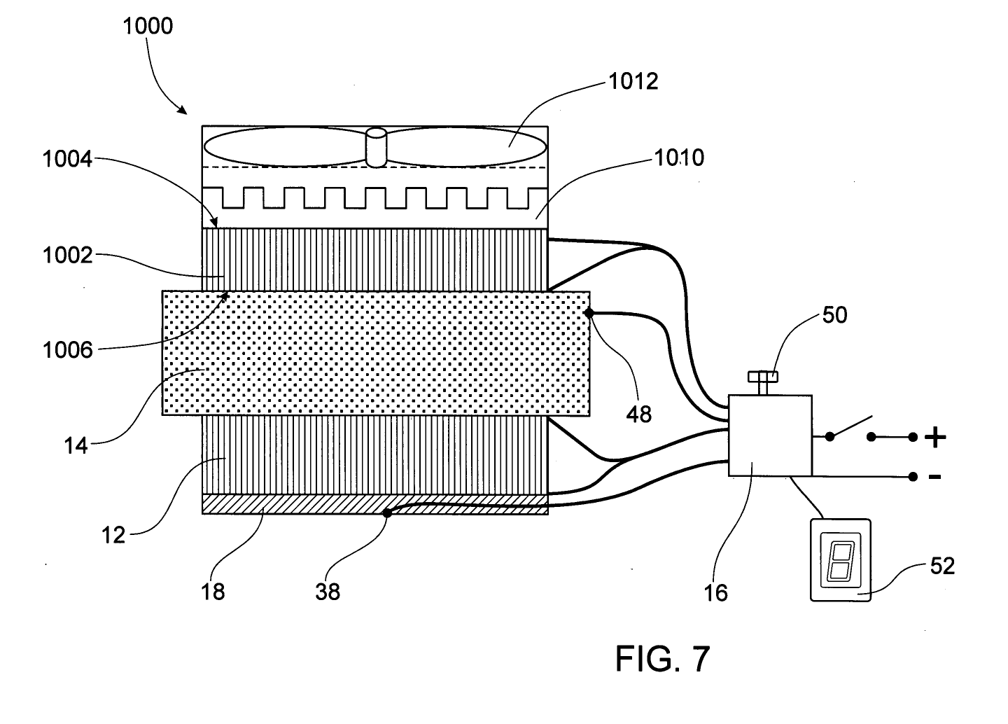

FIG. 7 shows yet another embodiment of a thermo-electric device 1000. The thermo-electric device 1000 is similar to the thermo-electric device 10. The same reference numerals are used to refer to features of the device 1000 which are the same as features of the device 10. The main difference between the device 1000 is and the device 10 is that the device 1000 includes a second Peltier module 1002 fixed to the thermal mass 14 to cool the thermal mass 14 to between 8° C. and 12° C.

The second Peltier module 1002 has a cold face 1006 and a hot face 1004. The cold face 1006 abuts the thermal mass 14 and is in thermal contact therewith. A heat sink 1010 is fixed to the hot face 1004 of the second Peltier module 42 to aid in dissipating heat from the hot face 1004. The device 1000 further includes a fan 1012 to cool down the heat sink 1010. The second Peltier module 1002 is powered from the control unit 16. In use, the second Peltier module 1002 is energised to cool the thermal mass 14 down to between 8° C. and 12° C. by heat exchange between the thermal mass 14 and the cold face 1006 of the second Peltier module 1002. The applicant envisages that in one embodiment the second Peltier module 1002 will be used to cool the thermal mass 14 before the test cycle only. In another embodiment the second Peltier module 1002 continuously cools the thermal mass 14 during the test cycle. In yet another embodiment of the invention, the thermal mass 14 is removably located so that the thermal mass 14 can be removed to be separately cooled and then replaced into abutment with the Peltier module 12. Thereafter the thermal mass 14 is further cooled with the second Peltier module 1002.

Thermally conductive paste or bonding agent is placed between the probe 18, Peltier module 12, thermal mass 14, second Peltier module 1002 and heat sink 1010 to increase heat transfer between the components.

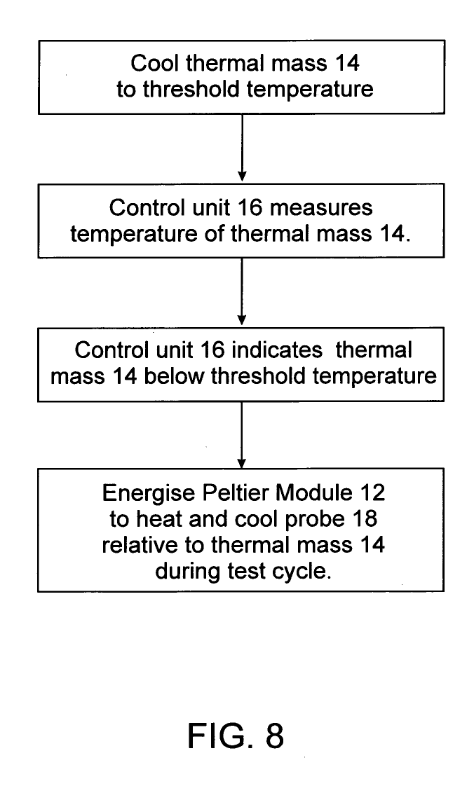

FIG. 8 shows a flow diagram of the method of thermal control of the device 10. The thermal mass 14 is cooled to the threshold temperature by contact with the cold block 30. Cooling may also be by the thermal mass 14 being placed in a freezer, as discussed, and then replaced into contact with the Peltier module 12. The control unit 16 measures the temperature of the thermal mass 14. If the temperature is measured to be below the threshold temperature, the LED 17 is energized to indicate that the device 10 it is ready for operating in the test cycle. Cooling of the thermal mass 14 is ceased before the test cycle by removing the device 10 from the cold block 30. The Peltier Module 12 is energised during cooling of the thermal mass 14 to have the probe 18 at the upper temperature limit, in anticipation of the test cycle. The probe is thereafter applied to the back of the neck of a patient and the start button 50 is pressed to start the test cycle wherein the Peltier Module 12 is energised to cool the probe 18 to the lower temperature limit.

Throughout the specification the aim has been to describe the invention without limiting the invention to any one embodiment or specific collection of features. Persons skilled in the relevant art may realize variations from the specific embodiments that will nonetheless fall within the scope of the invention. For example, the thermal mass may be cooled down to the threshold temperature by any of a number of cooling methods including, but not limited to, the conventional refrigeration cycle.

1.-34. (canceled)

35. A thermo-electric device comprising:
a probe;
a Peltier module having a probe side in thermal contact with the probe and an opposite interface side;
a control unit electrically connected to the Peltier module and connectable to a power source to energize the Peltier module so that the temperature of the probe side of the Peltier module, and thus the temperature of the probe, is variable in a range between a predetermined upper temperature limit and a predetermined lower temperature limit during a test cycle of the thermo-electric device; and
a thermal mass adapted for thermal contact with the interface side of the Peltier module and having thermal properties which enables the thermal mass to be cooled to a temperature below the predetermined upper temperature limit and thereafter maintain a temperature below the predetermined upper temperature limit for the duration of the test cycle.

36. The thermo-electric device of claim 35 wherein the control unit is configured to energise the Peltier module to heat the probe for part of the test cycle so that the probe is at a temperature warmer than the thermal mass, and to energise the Peltier module for a different part of the test cycle to cool the probe to a temperature cooler than the thermal mass.

37. The thermo-electric device of claim 35, wherein the control unit is configured to, during the test cycle, first energise the Peltier module to heat the probe relative to the thermal mass and then energise the Peltier module to cool the probe relative to the thermal mass.

38. The thermo-electric device of claim 35 wherein the control unit includes a temperature sensor at the thermal mass to measure the temperature of the thermal mass before the start of the test cycle.

39. The thermo-electric device of claim 35, wherein the control unit includes a visual or audible signal device to signal to a user of the thermo-electric device that the thermal mass has been cooled to below a threshold temperature, which is a temperature below the predetermined upper temperature limit.

40. The thermo-electric device of claim 39, wherein the control unit is configured to allow the thermo electric device to start the test cycle only if the temperature of the thermal mass is measured to be below the threshold temperature.

41. The thermo-electric device of claim 35 wherein the thermo-electric device includes a housing in which the thermal mass is located and at least part of the thermal mass is exposed to allow heat transfer of the thermal mass with a cold block during cooling of the thermal mass.

42. The thermo-electric device of claim 35 wherein the thermo-electric device includes a housing and the thermal mass is removably located in the housing.

43. The thermo-electric device of claim 35 wherein the thermo-electric device includes insulation surrounding at least part of the thermal mass.

44. The thermo-electric device of claim 35 wherein the thermal mass has a volumetric heat capacity of more than 2.00 J/cm³ K¹.

45. The thermo-electric device of claim 35 wherein the thermo-electric device includes a temperature sensor at the probe, the temperature senor being connected to the control unit.

46. The thermo-electric device of claim 35 wherein the thermo-electric device includes a secondary Peltier module having a cold face which is in thermal contact with the thermal mass and which brings the thermal mass to a temperature between the predetermined upper temperature limit and the predetermined lower temperature limit before the start of the test cycle.

47. The thermo-electric device of claim 35 wherein the thermo-electric device is a unitary handheld device.

48. The thermo-electric device of claim 35 wherein the thermo-electric device includes a rechargeable battery as the power source.

49. A thermo-electric system comprising the thermo-electric device of claim 35 and a base stand having a selectively cooled cold block which the thermal mass contacts during cooling of the thermal mass.

50. The thermo-electric system of claim 49, wherein the cold block has a spigot-shape and the thermal mass has a passage therein which is complementary-shaped to the cold block to receive the cold block.

51. A thermo-electric device kit comprising the thermo-electric device of claim 41 and a cold block in the form of a block of material on which the thermal mass is placed during cooling by heat transfer between the thermal mass and the block of material.

52. A method of thermal control of a thermo-electric device, the method including:
cooling a thermal mass of the thermo-electric device to a temperature below a predetermined upper temperature limit;
ceasing cooling of the thermal mass before the start of a test cycle of the thermo-electric device and for the duration of the test cycle; and
energising a Peltier module of the thermo-electric device so that a probe of the thermo-electric device, which is in thermal contact with the Peltier module, is variable between the predetermined upper temperature limit and a predetermined lower temperature limit during the test cycle.

53. The method of claim 52 wherein the thermal mass is cooled to a temperature between the predetermined upper temperature limit and the predetermined lower temperature limit before the test cycle.

54. The method of claim 52 wherein the Peltier module is energised to heat the probe for part of the test cycle so that the probe is at a temperature warmer than the thermal mass, and the Peltier module is energised for a different part of the test cycle to cool the probe to a temperature cooler than the thermal mass.

55. The method of claim 52 wherein, during the test cycle, the Peltier module is first energised to heat the probe relative to the thermal mass and then energised to cool the probe relative to the thermal mass.

56. The method of claim 52 wherein the thermal mass is cooled by putting the thermal mass into contact with a cold block so that the thermal mass is cooled by heat transfer between the thermal mass and the cold block.

57. The method of claim 52 wherein the thermal mass is located in a housing of the thermo-electric device and the thermal mass is removed from the housing for cooling and inserted back into the housing after cooling.

58. The method of claim 52 including measuring the temperature of the thermal mass before the start of the test cycle to determine whether the thermal mass is below a threshold temperature required for the test cycle.

59. The method of claim 52, wherein predetermined upper temperature limit is a temperature between 50 degrees Celsius and 25 degrees Celsius.

60. The method of claim 52 wherein the predetermined upper temperature limit is a temperature between 35 degrees Celsius and 25 degrees Celsius.

61. The method of claim 52, wherein the predetermined lower temperature limit is a temperature between 0 degrees Celsius and 8 degrees Celsius.

62. The method of claim 52 wherein the predetermined lower temperature limit is a temperature between 0 degrees Celsius and 5 degrees Celsius.

63. The method of claim 52 wherein the thermal mass is cooled to below a threshold temperature of 15 degrees Celsius before the start of the test cycle.
